(19) United States
(12) Patent Application Publication
(10) Pub. No.: US 2014/0046213 A1
(43) Pub. Date: Feb. 13, 2014

(54) DEVICE FOR ASSISTING WITH THE SAMPLING OF A BIOLOGICAL FLUID PRESENT IN A CAVITY

(71) Applicant: Marc Benbunan, Paris (FR)

(72) Inventor: Marc Benbunan, Paris (FR)

(73) Assignee: ASSISTANCE PUBLIQUE HOPITAUX DE PARIS, PARIS (FR)

(21) Appl. No.: 14/111,624

(22) PCT Filed: Apr. 13, 2012
(86) PCT No.: PCT/FR12/50817

§ 371 Date: Nov. 4, 2013

(30) Foreign Application Priority Data
Apr. 14, 2011 (FR) 1153229

(51) Int. Cl. A61B 5/15; A61B 10/00; A61B 5/153

(52) U.S. Cl. CPC A61B 5/150396; A61B 5/150038; A61B 5/153; A61B 10/0045
USPC 600/576

(57) ABSTRACT

A device for assisting with the sampling of a biological fluid present in a cavity, which device can co-operate with a puncture needle, the device including a holding member for keeping the device and the needle rigidly connected to one another while the needle is being inserted into the cavity; and a securing device which, following the introduction of the needle into the cavity, can be secured to an internal wall of the cavity.

### TECHNICAL FIELD OF THE INVENTION

The present invention relates to a device for assisting with the sampling of a biological fluid in a cavity, and more particularly, but not exclusively, of placental blood in an umbilical cord. The device is preferentially intended for cases in which the cavity is a non-innervated vessel such as an umbilical cord or instead a synthetic tube used in the laboratory. In fact, sampling using such a device is painful.

The technical field is, generally speaking, that of devices used to carry out samplings of a biological fluid. The device according to the invention is able to cooperate with a device for sampling a biological fluid. The invention will preferentially be described in the case where the device for sampling a biological fluid is a conventional puncture needle.

### TECHNOLOGICAL BACKGROUND OF THE INVENTION

Sampling placental blood from umbilical cords is a practice carried out increasingly over recent years, within the context of grafts of hematopoietic stem cells as alternative to the sampling of bone marrow, to reconstitute the haematopoiesis in patients suffering from haematological diseases. At present, to carry out a sampling of placental blood from an umbilical cord, an operator uses a conventional puncture needle, similar to those used for blood donations in blood transfusion establishments. This technique requires that the operator manually maintains the needle in the umbilical cord, throughout the duration of the blood collection, to prevent the needle from pulling out of the cord, but also to ensure a correct orientation of the needle in the cord, in order to guarantee a good collection of blood.

Given this constraint, the hands of the operator are immobilised and cannot carry out other manoeuvres capable of improving the collection of blood, such as a sub-pubic massage, a milking of the cord, or instead an agitation of the collection pouch.

Furthermore, statistical studies show that around 40% of blood samplings of umbilical cords cannot be used for stem cell grafts, because too little blood is collected.

### GENERAL DESCRIPTION OF THE INVENTION

The subject matter of the invention proposes a device for assisting with the sampling of a biological fluid present in a cavity, which can be more particularly used within the scope of sampling of placental blood in an umbilical cord, said device being installed on a puncture needle, improving the conditions for the collection of the biological fluid and enabling the operator to have available his two hands.

The invention thus essentially relates to a device for assisting with the sampling of a biological fluid present in a cavity, said device can cooperate with a puncture needle, said device being characterised in that it comprises:
a holding member for keeping the device and the needle rigidly connected to one another, while the needle is being inserted into the cavity;
securing means which, following the introduction of the needle into the cavity, can be secured to an internal wall of the cavity.

Thus, thanks to the invention, the needle is secured to the cavity via the securing means. Consequently, an operator carrying out a sampling of a biological fluid by means of needle having the device according to the invention, has available his two hands to carry out other manoeuvres.

Apart from the characteristics that have been evoked in the preceding paragraph, the device according to the invention may have one or more complementary characteristics among the following, considered individually or according to any technically possible combinations thereof:
The securing means are a plurality of fins secured on the holding member. A suitable arrangement of the fins thus makes it possible to. maintain spread apart the internal walls of the cavity, and consequently to improve the conditions for the collection of a biological fluid.
The holding member is a ring able to surround at least one portion of the needle while hugging the external shape of the needle. A good securement of the device on the needle is thereby assured.
The holding member and the securing means are made from metal. In this embodiment, the manufacture of the device and the handling to carry out to sample the biological fluid are easier.
The holding member and the securing means are made of plastic. In this embodiment, the risks of damaging the internal walls of the cavity are minimised.
When the holding member and the securing means are made of plastic, the device comprises maintaining means, and the securing means are able to take two positions:
   a retracted position according to which the securing means are maintained retracted by the maintaining means, such that they are able to be maintained against the internal wall of the needle.
   a deployed position according to which the securing means open out from the holding member during the removal of the maintaining means, such that they are able to come into contact with the internal wall of the cavity.

Thus the insertion of the needle provided with the device according to the invention is easier, because it is carried out with the securing means maintained in retracted position.
The maintaining means are a covering tube able to cover at least in part the outer wall of the puncture needle, said covering tube being able to move along the needle. The operator thus only has to slide the covering tube to make the securing means pass from the retracted position to the deployed position.
The holding member is a catheter able to be contained at least in part inside the needle, and to extend along the axis of the needle. Thus the introduction of the needle into the cavity is not hindered by the device.
The device comprises actuating means, and the securing means are able to take two positions:
   a retracted position according to which the securing means are maintained retracted such that they are able to be maintained against an internal wall of the needle,
   a deployed position according to which the securing means open out from the holding member, such that they are able to come into contact with the internal wall of the cavity, the passage from one position to another operating via the actuating means.

Thus the introduction of the needle into the cavity is not hindered by the securing means, which are then maintained in retracted position.
The actuating means are a connection piece secured to one end of the holding member, said connection piece being able to move along the axis of the needle. The actuating is then easy for the operator.

According to another aspect, the invention relates to a puncture needle comprising the device according to the invention.

The invention and its different applications will be better understood on reading the description that follows and on examining the figures that accompany it.

### BRIEF DESCRIPTION OF DRAWINGS

The figures are only presented by way of indication and in no way limit the invention. The figures show:

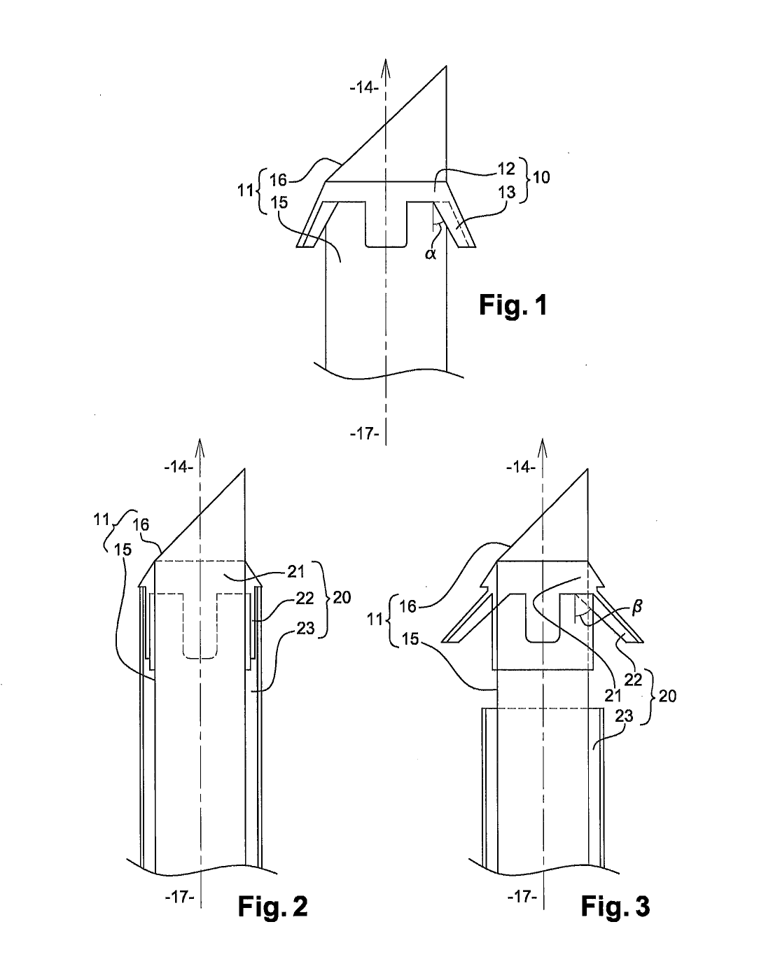

in FIG. 1, a schematic representation of a device for assisting with the sampling of a biological fluid according to a first embodiment of the invention, cooperating with a puncture needle;

in FIG. 2, a schematic representation of the device for assisting with the sampling of a biological fluid according to a second embodiment of the invention, before a puncture;

in FIG. 3, a schematic representation of the device of FIG. 2, following the puncture;

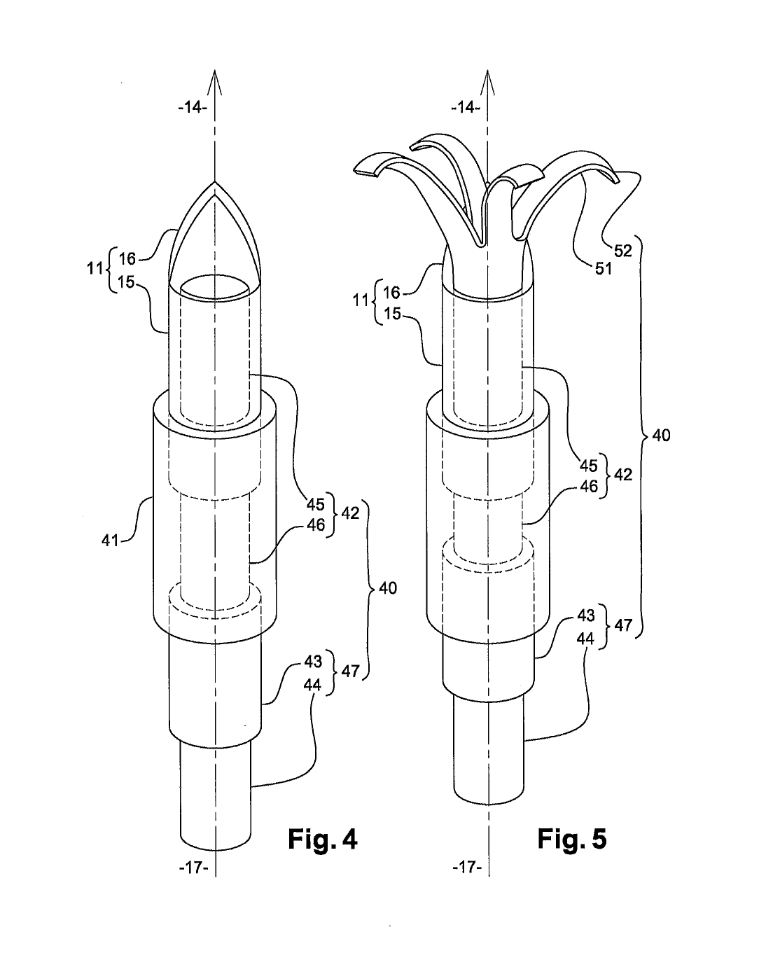

in FIG. 4, a schematic representation of the device for assisting with the sampling of a biological fluid according to a third embodiment of the invention, before a puncture;

in FIG. 5, a schematic representation of the device of FIG. 4, following the puncture.

### DETAILED DESCRIPTION OF AT LEAST ONE EMBODIMENT OF THE INVENTION

Unless stated otherwise, a same component appearing in different figures has a single reference.

Throughout the description that follows, and with reference to FIGS. 1 to 5, the terms “front” and “rear” are to be considered with respect to a direction of advancement of a puncture needle 11 encountered following the introduction of the needle 11 into a cavity by an operator, this direction being schematically represented by an arrow 14.

In a known manner, the needle 11 comprises a cylindrical part 15 and a point 16. The cylindrical part 15 extends along an axis 17, and is surmounted by the point 16. Conventionally, the cylindrical part 15 of the needle 11 is partially fitted into a needle support 41, represented in FIGS. 4 and 5, and not visible in FIGS. 1 to 3 since said needle support 41 is situated relatively to the rear of the needle 11. FIGS. 1 to 3 only in fact show a front part of the needle 11. The needle support 41 enables the operator to hold the needle 11. In the non-limiting embodiment described, the needle support 41 is of cylindrical shape and extends along the axis 17. The shape of the needle support 41 hugs the shape of the cylindrical part 15 of the needle 11.

FIG. 1 is a schematic representation of a device for assisting with the sampling of a biological fluid 10 in the cavity, said device 10 cooperating with the needle 11, according to a first embodiment of the invention.

The device 10 comprises:
a holding member 12
securing means 13

In this first embodiment:
The holding member 12 is a ring 12 extending along the axis 17, and the shape of which hugs the shape of the cylindrical part 15 of the needle 11. The ring 12 surrounds a portion of the cylindrical part 15 of the needle 11. The height of the ring 12 is of the order of several millimetres, for example one millimetre. It should be noted that the ring 12 is in direct contact with the cylindrical part 15 of the needle 11.
The securing means 13 are fins 13. They are four in number, of which three are visible in FIG. 1, but according to other embodiments of the invention, the number of fins 13 may be different. The length of the fins 13 is of the order of several millimetres, for example two millimetres. The fins 13 open out from the ring 12 towards the rear, in other words that they form an angle α less than 90° with the surface of the needle 11, and extend in the direction opposite to the point 16 of the needle 11. α is advantageously comprised between 25° and 35°.

Such an opening out of the fins 13 enables an introduction of the needle 11 into the cavity, but prevents a removal of the needle 11 from the cavity. In fact, the fins 13 open out towards the rear, they prevent a movement of removal of the needle 11 according to the principle of the harpoon: the fins 13 are secured to the internal walls of the cavity. It may be noted that the fins 13 are advantageously. constituted of a sufficiently flexible material so that the angle α can vary by several degrees, thereby facilitating the introduction of the needle 11 into the cavity, but sufficiently rigid to prevent the fins 13 from turning back towards the rear of the needle 11, in other words that the angle does not become greater than 90°. In fact, a turning back of the fins 13 would enable a withdrawal of the needle 11 from the cavity, yet the device 10 is adapted to remain secured to the internal walls of the cavity following the introduction of the needle 11. The device 10 is in fact preferentially dedicated to the sampling of placental blood from umbilical cords, said umbilical cords being non-innervated and condemned to be cut following the sampling.

According to an embodiment of the invention, the fins 13 and the ring 12 are made from metal. The needle 11, the ring 12 and the fins 13 are for example made from a single piece, or instead secured to each other by welding. According to another embodiment of the invention, the fins 13 and the ring 12 are made from plastic. The ring 12 is then for example secured to the needle 11 by bonding.

Furthermore, the ring 12 is advantageously installed as far forward as possible from the cylindrical part 15 of the needle 11. In a preferred embodiment, the fins 13 are installed at a distance comprised between eight and ten millimetres from the point 16 of the needle 11. Thus, because of their opened out shape, the fins 13 spread apart the internal walls of the cavity as close as possible to the collection of biological fluid by the needle 11. The collection of a biological fluid is thereby improved.

FIGS. 2 and 3 are schematic representations of a device for assisting with the sampling of a biological fluid 20 in the cavity, said device 20 cooperating with the needle 11, according to a second embodiment of the invention.

The device 20 comprises:
a holding member 21
securing means 22
maintaining means 23

In this second embodiment, the holding member and the securing means are made of plastic, and:
The holding member 21 is a ring 21 extending along the axis 17, and the shape of which hugs the shape of the cylindrical part 15 of the needle 11. The holding member 21 surrounds a portion of the cylindrical part 15 of the needle 11.
The securing means 22 are fins 22 rigidly connected to the ring 21.

The fins 22 are able to take two positions:
a retracted position according to which the fins 22 are maintained against the cylindrical part 15 of the needle 11 by the maintaining means 23. This retracted position is represented in FIG. 2.
a deployed position according to which the fins 22 open out from the ring 21 towards the rear according to an angle β, such that they are able to come into contact with the internal wall of the cavity. This deployed position is represented in FIG. 3.
   The maintaining means 23 are a covering tube 23 made of plastic, of cylindrical shape, and extending along the axis 17. The shape of the covering tube 23 hugs the shape of the cylindrical part 15 of the needle 11. The covering tube 23 is able to move along the cylindrical part 15 of the needle 11. In the retracted position, the covering tube 23 covers the fins 22, maintaining them pressed against the cylindrical part 15 of the needle 11. The operator thus easily inserts the needle 11 into the cavity. Then the operator only has to slide the covering tube 23 towards the rear, to allow the fins 22 to pass from the retracted position to the deployed position. The needle 11 then can no longer be removed from the cavity, the fins 22 being secured to the internal walls of the cavity according to the principle of the harpoon.

The device 20 according to the second embodiment of the invention is more complicated to manufacture and to handle than the device 10 according to the first embodiment of the invention, but the introduction of the needle 11 into the cavity is easier, and the risks of creating lesions on the internal walls of the cavity are minimised.

FIGS. 4 and 5 are schematic representations of a device for assisting with the sampling of a biological fluid 40 in the cavity, said device 40 cooperating with the needle 11, according to a third embodiment of the invention.

The device 40 comprises:
a holding member 42
securing means 51
actuating means 47

In this third embodiment:
the holding member 42 is a catheter 42. The catheter 42 is of cylindrical shape and extends along the axis 17. The catheter 42 comprises an upper part 45 contained in the needle 11, and a lower part 46 contained in the needle support 41. The catheter 42 is able to move along the axis 17, via the actuating means 47.
The securing means 51 are fins 51. They are four in number, but according to other embodiments of the invention, the number of fins may be different.
The actuating means 47 comprise a connection piece 43 and a screwing piece 44. The connection piece 43 and the screwing piece 44 are of cylindrical shape and extend along the axis 17. The connection piece 43 is secured on one side to the lower part 46 of the catheter 42, and on another side to the screwing piece 44. The connection piece 43 is able to move by screwing along the screwing piece 44, carrying along in its movement the catheter 42 to which it is rigidly connected.

The fins 51 are able to take two positions:
a retracted position according to which the fins 51 are maintained against an internal wall of the needle 11. This retracted position is represented in FIG. 4.
a deployed position according to which the fins 51 open out from the catheter 42 towards the front, in other words that they form an angle Γ below 90° with the surface of the needle 11, and extend in the direction of the point 16 of the needle 11. In deployed position, the fins 51 come into contact with the internal wall of the cavity. This deployed position is represented in FIG. 5. Each fin 51 has an end 52 bent back towards the rear, such that the fin 51 is able to secure itself to the internal wall of the cavity according to the principle of the harpoon.

The passage from one position to another takes place via the actuating means 47: by screwing towards the front the connection piece 43 along the screwing piece 44, the operator causes a movement of the catheter 42 towards the rear. The fins 51 then go from the retracted position to the deployed position, on removing the needle 11. The fins 51 are then secured to the internal wall of the cavity, thereby preventing a removal of the needle 11 outside of the cavity. It may be noted that the passage from one position to the other takes places without opening of the sampling circuit, which enables a sterile sampling of the biological fluid. Furthermore, because of their opened out shape, the fins 51 spread apart the internal walls of the cavity thereby improving the collection of a biological fluid.

1. Device for assisting with the sampling of a biological fluid present in a cavity, said device can constructed and arranged to cooperate with a puncture needle, said device comprising:
a holding member for keeping the device and the needle rigidly connected to one another, while the needle is being inserted into the cavity;
a securing device which, following the introduction of the needle into the cavity, can be secured to an internal wall of the cavity.

2. Device according to claim 1, wherein the securing device includes a plurality of fins secured on the holding member.

3. Device according to claim 1 wherein the holding member is a ring able to surround at least one portion of the needle while hugging the external shape of the needle.

4. Device according to claim 1, wherein the holding member and the securing device are made from metal.

5. Device according to claim 1, wherein the holding member and the securing device are made of plastic.

6. Device according to claim 1, comprises comprising a maintaining device, and wherein the securing device is able to take two positions:
a retracted position according to which the securing device is maintained retracted by the maintaining device, such that the securing device is able to be maintained against the internal wall of the needle.
a deployed position according to which the securing device opens out from the holding member during the removal of the maintaining device, such that the securing device is able to come into contact with the internal wall of the cavity.

7. Device according to claim 6, wherein the maintaining device includes a covering tube able to cover at least in part the outer wall of the needle, said covering tube being able to move along the needle.

8. Device according to claim 1, wherein the holding member is a catheter able to be contained at least in part inside the needle, and to extend along the axis of the needle.

9. Device according to claim 8, comprising an actuator, and wherein the securing device is able to take two positions:
a retracted position according to which the securing device is maintained retracted such that the securing device is able to be maintained against an internal wall of the needle,
a deployed position according to which the securing device opens out from the holding member, such that the securing device is able to come into contact with the internal wall of the cavity,
the passage from one position to another operating via the actuator.

10. Device according to claim 9, wherein the actuator includes a connection piece secured to one end of the holding member, said connection piece being able to move along the axis of the needle.

11. Puncture needle comprising the device according to claim 1.
